(19) United States
(12) Patent Application Publication
(10) Pub. No.: US 2010/0093083 A1
(43) Pub. Date: Apr. 15, 2010

(54) LARGE SCALE PRODUCTION OF STEM CELLS

(76) Inventors: Hazel Thompson, Welwyn (GB); Julie Kerby, Welwyn Garden City (GB)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/527,626

(22) PCT Filed: Feb. 18, 2008
(86) PCT No.: PCT/GB08/00558

§ 371 Date: Dec. 3, 2009

(30) Foreign Application Priority Data
Feb. 19, 2007 (GB) 0703188.3
Apr. 10, 2007 (GB) 0706917.2

(51) Int. Cl. C12N 5/0735

(52) U.S. Cl. 435/366

(57) ABSTRACT

Methods for large-scale production of stem cells, including embryonic stem cells, are provided. Also provided are methods for large-scale production of differentiated cells derived from stem cells and use of stem cells or the differentiated progeny thereof in assays.

The present invention relates to methods for large-scale production of stem cells, including pluripotent and embryonic stem cells. The invention also relates to methods for large-scale production of differentiated cells derived from stem cells in culture. The invention also relates to the use of stem cells or the differentiated progeny thereof in assays, for example for drug discovery.

The establishment and maintenance of stem cell cultures in vitro, including cultures of pluripotent stem cells such as embryonic stem (ES) cells, is well known. For example, the culture of pluripotent stem cell cultures in the presence of medium containing serum and Leukaemia Inhibitory Factor (LIF) is described in Smith et al. (1988) Nature 336: 688-90. Stem cell cultures can also be induced to differentiate in vitro and hence provide a source of differentiated cell types, including progenitor and stem cells, which are otherwise difficult to obtain. For example, mouse ES cell lines can be expanded in vitro and have the ability to give rise to cells from all three germ layers (Smith (2001) Annu Rev Cell Dev Biol 17: 435-462).

The increasing availability of stem cell lines has led to an increased awareness of the value and potential of such cell lines for research and for potential therapeutic applications. For example, stem cell lines and specific differentiated cell types can offer the drug discovery community a cell culture model that is more physiologically relevant than immortalised cell lines and that avoids the expense and time of in vivo experiments and the development of animal models. However, if stem cells are to be widely adopted as research tools it will be necessary to develop culture processes for stem cell growth and differentiation that reliably and reproducibly yield the quantity and quality of cells required. In the case of high throughput screening assays in which millions of compounds are screened, the number of cells required would be in the order of billions of cells per day. Production of stem cells in such quantities using conventional bench scale culture methods is not attractive in terms of cost or in terms of the potential variability of output. Moreover, it is essential that the culture conditions do not result in spontaneous differentiation of the stem cells or any loss of differentiation potential. Thus, there is a requirement for both reproducible expansion of stem cell populations and reproducible control of the phenotype of the cells within the expanded cultures. Current multiple parallel manual culture processes fail to satisfy these criteria.

Previous attempts to scale up the production of stem cells have failed to achieve reproducible production of the required numbers of stem cells and/or have encountered difficulties in scale-up which have not been addressed.

For example, Fok and Zandstra ((2005) Stem Cells 23: 1333-1342) describe 50 ml cultures of mouse ES cells on microcarriers in 100 ml spinner flasks. However, such cultures exhibited increased population doubling times and reduced cumulative fold expansion when compared to tissue culture flask controls. In addition, significant “bead-bridging” (i.e. formation of aggregates of microcarriers and cells) was acknowledged to lead to creation of a suboptimal culture environment.

Scaled-up cultures of neural precursor cell aggregates have been attempted in computer-controlled bioreactors (Gilbertson et al. (2006) Biotechnology and Bioengineering 94: 783-792). However, it has not been established whether such techniques are applicable to other types of stem cells.

Controlled differentiation of stem cells into specific types of differentiated cells has not been reported on a scale appropriate for commercial applications. In particular, production of an expanded population of differentiated cells derived from a relatively small population of stem cells has not been reported.

Thus, there remains a need for robust and reproducible methods for large-scale expansion and differentiation of stem cells. Accordingly, objects of the invention include the provision of methods for large-scale production of stem cells or of differentiated cells derived from stem cells. Another object of the invention is the provision of populations of stem cells, or differentiated cells derived therefrom, which can be used in assays.

Accordingly, a first aspect of the invention provides a method for large-scale production of pluripotent stem cells, suitably embryonic stem (ES), embryonic carcinoma (EC), embryonic gonadal (EG) cells, induced pluripotent stem (iPS) cells or reprogrammed cells, comprising:
a) inoculating a first volume of serum-free culture medium containing a plurality of microcarriers with stem cells;
b) allowing the stem cells to adhere to the microcarriers;
c) adding a second volume of serum-free medium to the culture; and
d) incubating the culture under conditions conducive to the proliferation of the stem cells.

The invention also provides a method for large-scale production of stem cells other than mouse ES cells comprising:
a) inoculating a first volume of culture medium containing a plurality of microcarriers with stem cells;
b) allowing the stem cells to adhere to the microcarriers;
c) adding a second volume of medium to the culture; and
d) incubating the culture under conditions conducive to the proliferation of the stem cells.

Such methods may be used to culture any type of stem cell that is of interest including mouse stem cells and human stem cells. Other mammalian stem cells can also be cultured according to the methods of the invention, including rat, American mink, hamster, pig, sheep, cow and primate stem cells.

It is intended, for the purposes of the present invention, that the term stem cell embraces any cell having the capacity for self-renewal and the potential to differentiate into one or more other cell types. Thus, the term stem cell includes pluripotential, multipotential or unipotential stem cells and progenitor cells from any tissue or stage of development. For example, the desired stem cells may be ES cells, EC cells, EG cells, iPS cells, reprogrammed cells, haematopoietic stem cells, epidermal stem cells, mesenchymal stem cells, adipose tissue-derived stem cells, muscle stem cells or neural stem cells.

The methods described herein are suitable for use with media containing serum and with serum-free media. However, it is preferred that the culture medium is serum-free. The replacement of serum or other incompletely defined or undefined medium components with defined medium components can also result in greater reproducibility of the methods of the invention.

A number of suitable culture media, including serum-free media, are commercially available for the culture of stem cells and are suitable for use in the methods of the invention. Typically the medium used to expand populations of stem cells according to the methods of the invention will be the same medium used for conventional culture in plates or tissue culture flasks.

However, in some embodiments a different medium may be used in the methods of the invention of the medium may be supplemented with additional components that promote proliferation or survival of the stem cells and/or prevent differentiation. For example, the medium may, in some embodiments, comprise a combination of a MEK inhibitor, a GSK3 inhibitor and, optionally, an antagonist of an FGF receptor. This combination is the subject of a co-pending patent application, GB0615327.4, filed on 1 Aug. 2006, the contents of which are incorporated herein by reference.

Reference to GSK3 inhibition refers to inhibition of one or more GSK3 enzymes. The family of GSK3 enzymes is well-known and a number of variants have been described (see e.g. Schaffer et al.; Gene 2003; 302 (1-2): 73-81). In specific embodiments GSK3-β is inhibited. GSK3-α inhibitors are also suitable, and in general inhibitors for use in the invention inhibit both. A wide range of GSK3 inhibitors are known, by way of example, the inhibitors CHIR 98014, CHIR 99021, AR-A0144-18, TDZD-8, SB216763 and SB415286. Other inhibitors are known and useful in the invention. In addition, the structure of the active site of GSK3-β has been characterised and key residues that interact with specific and non-specific inhibitors have been identified (Bertrand et al.; J Mol Biol. 2003; 333(2): 393-407). This structural characterisation allows additional GSK inhibitors to be readily identified.

The inhibitors of certain embodiments are specific for GSK3-β and GSK3-α, substantially do not inhibit erk2 and substantially do not inhibit cdc2. Preferably the inhibitors have at least 100 fold, more preferably at least 200 fold, very preferably at least 400 fold selectivity for human GSK3 over mouse erk2 and/or human cdc2, measured as ratio of IC₅₀ values; here, reference to GSK3 IC₅₀ values refers to the mean values for human GSK3-β and GSK3-α. Good results have been obtained with CHIR 99021 and CHIR 98014, which are both specific for GSK3. Examples of GSK3 inhibitors are described in Bennett C, et al, J. Biol. Chem., vol. 277, no. 34, Aug. 23, 2002, pp 30998-31004 and in Ring D B, et al, Diabetes, vol. 52, March 2003, pp 588-595. Suitable concentrations for use of CHIR 99021 are in the range 0.01 to 100, preferably 0.1 to 20, more preferably 0.3 to 10 micromolar.

Reference to a MEK inhibitor herein refers to MEK inhibitors in general. Thus, reference to a MEK inhibitor refers to any inhibitor a member of the MEK family of protein kinases, including MEK1, MEK2 and MEK3. Reference is also made to MEK1, MEK2 and MEK3 inhibitors. Examples of suitable MEK inhibitors, already known in the art, include the MEK1 inhibitors PD184352 and PD98059, inhibitors of MEK1 and MEK2 U0126 and SL327, and those discussed in Davies et al (2000) (Davies S P, Reddy H, Caivano M, Cohen P. Specificity and mechanism of action of some commonly used protein kinase inhibitors. Biochem J. 351, 95-105). In particular, PD184352 has been found to have a high degree of specificity and potency when compared to other known MEK inhibitors. Other MEK inhibitors and classes of MEK inhibitors are described in Zhang et al. (2000) Bioorganic & Medicinal Chemistry Letters; 10:2825-2828.

Inhibition of MEK kinases can also be conveniently achieved using RNA-mediated interference (RNAi). Typically, a double-stranded RNA molecule complementary to all or part of a MEK gene is introduced into pluripotent cells, thus promoting specific degradation of MEK-encoding mRNA molecules. This post-transcriptional mechanism results in reduced or abolished expression of the targeted MEK gene. Suitable techniques and protocols for achieving MEK inhibition using RNAi are known.

A number of assays for identifying kinase inhibitors, including GSK3 inhibitors and MEK inhibitors, are known. For example, Davies et al (2000) describe kinase assays in which a kinase is incubated in the presence of a peptide substrate and radiolabelled ATP. Phosphorylation of the substrate by the kinase results in incorporation of the label into the substrate. Aliquots of each reaction are immobilized on phosphocellulose paper and washed in phosphoric acid to remove free ATP. The activity of the substrate following incubation is then measured and provides an indication of kinase activity. The relative kinase activity in the presence and absence of candidate kinase inhibitors can be readily determined using such an assay. Downey et al. (1996) J Biol Chem.; 271(35): 21005-21011 also describes assays for kinase activity which can be used to identify kinase inhibitors.

It is preferred that optimum culture conditions are maintained throughout the culture as, for example, sub-optimal conditions can induce spontaneous differentiation of pluripotent cells. Thus, it is preferred that the culture of step d) is subjected to agitation in order to avoid the development of localised regions of sub-optimal culture conditions, for example due to microcarriers settling at the bottom of the culture vessel. Agitation of the culture medium also advantageously allows the stem cells to be cultured at higher densities than is possible in tissue culture flasks. In particular, the use of microcarriers provides an increased surface area for cell growth whilst agitation ensures that there is adequate distribution of nutrients and oxygen to all cells in the culture. In some embodiments of the invention it has been found that 1 L of culture can yield as many stem cells as 70 to 100 T-175 tissue culture flasks maintained using conventional methods.

Agitation can be achieved in a number of ways, including the use of spinner flasks containing a magnetic paddle or impeller. Alternatively, the methods of the invention can be carried out in a bioreactor. A number of types of bioreactor are available, including bioreactors in which agitation of the medium is achieved using a paddle or impeller and rotary wall bioreactors. Rotary wall bioreactors can additionally be used to simulate conditions of reduced gravity (microgravity) which, in some embodiments, may promote desirable cell growth or differentiation characteristics.

An advantage of using a bioreactor is that one or more culture parameters can be monitored and/or controlled, either continuously or at intervals determined by the user. The monitoring can be carried out using probes inserted into the culture vessel. Alternatively or additionally, a medium sample can be withdrawn from the culture vessel and analysed. Typically, one or more of the oxygen concentration, the pH, the concentration of glucose, the concentration of lactate, and the shear rate are controlled. This allows optimum or near-optimum culture conditions to be maintained spatially and temporally for the duration of the culture, for example by varying one or more culture parameters.

A number of parameters are important for maintaining and expanding stem cells in large-scale cultures, including temperature, oxygen concentration, pH, concentrations of nutrients (e.g. glucose) and waste products (e.g. lactate), and shear rate. In general, the temperature at which mammalian stem cells are cultured is about 37° C.±0.5° C., although the skilled person will appreciate that wider variations of temperature might be possible in some embodiments. Typically, temperature is controlled by placing the culture vessels in temperature-controlled incubators or, in the case of some bioreactors, by direct monitoring and control of the temperature in the culture vessel. Temperature variations can be minimised by ensuring all media and culture components are brought to the required temperature before being added to the culture vessel.

The provision and distribution of oxygen to all cells within the culture is another important factor in maintaining healthy populations of stem cells, although the specific oxygen requirements will vary between different cell types. In particular, as the culture volume increases it becomes increasingly difficult to ensure adequate and even supply of oxygen to the whole culture. In some embodiments of the invention, oxygen supply is increased by means of agitation of the medium, e.g. as described herein, to increase the amount of oxygen dissolved in the medium. Typically, the source of oxygen will be the headspace of the culture vessel, and the rate of agitation will be selected so as to ensure adequate oxygen supply to the cells without generating a shear rate that damages or impairs the growth of the cells. Gas exchange in the headspace is generally achieved by direct supply of gas to the culture vessel or by use of vented culture vessels in gassed incubators. In both cases, the gas supply is usually approximately 5% CO₂ in air. Some variation in the proportion of CO₂ in the gas is possible, e.g. +/−1% or +/−2%. Other methods for supplying oxygen to stem cell cultures of the invention are also possible, e.g. sparging. In particular, oxygen can be passed through gas permeable tubing into the liquid in the bioreactor, thus minimising damage to the cells due to the formation of bubbles during sparging.

The pH of the culture medium can also be monitored and/or controlled in order to maintain optimal culture conditions. Control of pH can be achieved by any suitable method, including replacement of all or a portion of the culture medium or direct addition of pH-adjusting agents to the medium. pH is commonly controlled by gassing the headspace or the culture media with 5-7% CO₂ in air.

Similarly, the concentrations of nutrients and/or waste products can be monitored and/or controlled. For example, in some embodiments glucose concentration is monitored to provide an indication of the nutrients available to the stem cells. In other embodiments lactate concentration is monitored to provide an indication of the levels of waste products in the medium that might reduce cell survival, growth or proliferation. The presence of nutrients and waste products in the culture medium can be controlled by replacement of all or a portion of the culture medium. Nutrients can also be provided by the addition of specific medium components or additional medium to the stem cell cultures.

It is also desirable to monitor and/or control the shear forces experienced by cells in operation of the methods of the invention. As discussed above, optimal conditions in cultures subjected to agitation require balancing the requirement for even distribution of oxygen and nutrients throughout the culture against the need to avoid cell damage due to excessive shear forces. In certain embodiments of the invention, shear forces are controlled by adjusting the stirring rate (e.g. by reducing or increasing the speed of the paddle or impeller). The shear rate can also be used to control the aggregation of cells within the cultures. For example, in some embodiments it is preferred that the shear rate is increased with time in culture to compensate for the increasing weight of the microcarriers caused by proliferation of attached cells. This advantageously counters the tendency of the microcarriers to settle under gravity and avoids excess shear at early stages of the culture, thus maintaining optimum culture conditions and reducing or eliminating the formation of aggregates of microcarriers and cells.

In certain embodiments of the invention all or a proportion of the culture medium is periodically replaced with fresh medium in order to maintain preferred culture conditions. Thus, step d) of the method preferably comprises periodic replacement of a proportion of the medium volume with fresh medium. The proportion of medium volume replaced will vary between different embodiments of the invention and may be 10%, 20%, 30%, 40%, 50%, 60%, 70%, 80% or 90% of the culture volume. The frequency of medium replacement can also vary, and may be, for example, every 12, 24, 36 or 48 hours. The precise proportions and frequencies chosen in different embodiments will depend on the type of cells being cultured, the culture medium, the type of culture vessel, and other culture parameters, and can be readily determined by the user.

Typically the total culture volume will be in excess of about 100 ml, 250 ml, 500 ml or 1000 ml. However, the maximum culture volume is in principle without limit, provided that culture parameters such as oxygen concentration and pH are controlled. For example, bioreactors having a culture capacity of 70,000 L are known. It is noted that the total volume of the culture vessel will, in general, significantly exceed the culture volume. In specific examples described herein, the total culture volume is approximately half of the total volume of the culture vessel. The excess volume in the culture vessel forms a headspace which provides a source of oxygen, as described above.

In some embodiments of the invention, the medium is continually replenished, i.e. fresh medium is continually added and used medium is continually removed. In such continuous perfusion cultures, it is desirable to establish a steady state in which one or more culture parameters are maintained at an essentially constant level, or within a permitted range, for the duration of the culture. Continuous perfusion methods of the invention will typically monitor the concentration of glucose or lactate in the culture medium and vary the rate of medium replacement in order to keep the glucose or lactate concentration at a value or within a range determined by the user. In some embodiments other parameters or combinations of parameters described herein may be used to control the rate of medium replacement. For stem cells in particular, it may be beneficial to maintain levels of growth factors or cytokines which can supplement the media and contribute to maintenance of the stem cell phenotype.

The stem cells are typically inoculated into a relatively small first volume of medium containing microcarriers, and the medium volume increased to the total culture volume after the stem cells have adhered to the microcarriers. For example, the first volume can be about 5%, 10%, 20%, 30%, 40% or 50% of the total (final) culture volume. In some embodiments, satisfactory results can be obtained when the first volume is equal to the total culture volume, i.e. without increasing the culture volume after the stem cells have been allowed to adhere to the microcarriers.

Preferably, the first volume of culture medium is inoculated with pluripotent cells or other stem cells in suspension. The suspension may comprise single cells or small clusters of stem cells. Typically, the clusters of stem cells will each contain from about 10 to about 20 or 30 cells. For some stem cell types it is preferred that the inoculated stem cells are not in single cell suspension as this leads to reduced survival of the stem cells in subsequent culture. For some types of stem cell, including some pluripotent human cells, it is found that dissociation into single cells tends to lead to cell death. However, survival in subsequent culture is significantly improved if clumps of stem cells are used to inoculate the cultures.

Periodic or continuous agitation of the first volume of culture medium may be carried out whilst the stem cells are being permitted to adhere to the microcarriers to aid uniform distribution of the stem cells across the microcarriers.

The number of stem cells inoculated will depend on the type of stem cell and the total final culture volume. In the specific examples described herein, stem cells have been inoculated at 5×10⁴ 1×10⁵ cells/ml final culture medium and successfully expanded using the methods of the invention.

The production of stem cells according to the invention is most effective when the initial population of stem cells inoculated into the culture medium is of high quality. It is preferred that the stem cells for inoculation are not confluent and are in log phase growth. The cultures of stem cells from which the inoculum is obtained should therefore be passaged sufficiently in advance of inoculation to ensure that the cells are in good condition. The precise timing will depend on the type of stem cell and the doubling time obtained in the particular culture conditions used. Typically, for mouse ES cells, passaging the cells 24 hours in advance of inoculation will result in non-confluent cells in log phase growth.

A number of types of microcarriers are commercially available and suitable for use in the methods of the invention, including Cytodex 1, Cytodex 3 (Amersham Biosciences), 2D Microhex (Nunc) and Cultispher (Percell Biolytica). Microcarriers may be made of tissue culture plastic, dextran or gelatin and may be coated with collagen or gelatin. The microcarriers may be supplied pre-coated or the coating can be applied by the user using standard methods. In some applications of the invention, particular types of microcarriers will be particularly suitable, e.g. biocompatible, biodegradable and/or animal component-free microcarriers. Generally, microcarriers have diameters of 1 mm or less, preferably 500 μm or less and often in the range 30-300 μm

In use, the microcarriers can be processed according to the manufacturer's directions. Typically, the microcarriers will be sterilised and washed in buffer and/or culture medium before the cultures of the invention are set up. In preferred embodiments, the microcarriers are sterilised by autoclaving in phosphate buffered saline (PBS) and, if necessary, stored at 4° C. until required. Before use, the microcarriers are washed in fresh PBS and then washed or preferably soaked in culture medium at 37° C., typically for about 1 hour. In the hands of the present inventors, soaking the microcarriers in medium prior to use has yielded particularly good results.

The density of microcarriers used in cultures carried out using the methods of the invention will vary according to the type of microcarrier that is used and the cells that are cultured. Typical microcarrier densities, e.g. as used in the specific examples, are about 0.5±1 g/litre (Cultispher microcarriers) and about 1 g/litre (Cytodex microcarriers).

Following expansion of the stem cells using the methods of the invention, it is preferred that the additional step of e) harvesting the stem cells is carried out. In some embodiments, there is no need to separate the stem cells from the microcarriers and the microcarriers are simply separated from the culture medium. This can be achieved by any suitable method, e.g. filtration. However, it is particularly convenient to allow the microcarriers and attached cells to settle to the bottom of the culture vessel in the absence of agitation, decant or aspirate off surplus culture medium, and transfer the microcarriers to an appropriate vessel.

In some embodiments, the harvesting comprises a) isolating the microcarriers from the culture medium; and b) separating the stem cells from the microcarriers. The stem cells can be separated from the microcarriers using an enzymatic or non-enzymatic cell dissociation reagent. Suitable cell dissociation reagents include trypsin-EDTA, Accutase (Chemicon) and Cell Dissociation Buffer (Invitrogen). In some embodiments, the microcarriers are soluble in the cell dissociation reagent, thus enabling convenient isolation of the stem cells without the need to remove the microcarriers in an additional step. For example, gelatin microcarriers are soluble in trypsin-EDTA.

The methods of the invention are suitable for production of feeder-dependent stem cells as well as feeder-independent/feeder-free cultures. This is of particular value for expanding feeder-dependent human ES cell lines, as described in the specific examples, although other feeder-dependent stem cells can be cultured according to the invention. If feeder cells are to be used, the feeder cells are inoculated into medium containing a plurality of microcarriers and are permitted to adhere to the microcarriers prior to the inoculation of step a). Preferably, the feeder cells are permitted to proliferate until confluence prior to the inoculation of step a). It is also preferred that the feeder cells are inactivated prior to the inoculation of step a), for example using known protocols for γ-irradiation or mitomycin c treatment.

In a second aspect, the methods of the invention can also be adapted to permit the large-scale production of differentiated cells derived from stem cells. Such methods advantageously permit the production of large, and potentially limitless, numbers of cells of a desired phenotype by means of in vitro differentiation of stem cells. Potentially any cell type, even cell types that are rare in vivo, can be provided reproducibly and in large numbers.

Thus, the invention provides a method for large-scale production of desired differentiated cells derived from stem cells comprising:
a) providing a suspension of particles comprising the stem cells adhered to microcarriers in culture medium; and
b) inducing differentiation of the stem cells on the microcarriers.

In a related third aspect, the invention provides a method for large-scale production of desired differentiated cells derived from stem cells comprising:
a) inoculating a first volume of culture medium containing a plurality of microcarriers with stem cells;
b) allowing the stem cells to adhere to the microcarriers;
c) adding a second volume of medium to the culture; and
d) incubating the culture under conditions conducive to the differentiation of the stem cells into the desired differentiated cells.

In preferred embodiments of the second and third aspects of the invention, the culture medium is serum-free.

The methods of the invention can be used to produce large populations of multipotential or unipotential stem cells and terminally differentiated cells of a given phenotype by directing differentiation of the inoculated stem cells along one or more lineage pathways. Thus, the differentiated cells can for example be somatic stem cells, haematopoietic stem cells, epidermal stem cells, mesenchmal stem cells, adipose tissue-derived stem cells, muscle stem cells or neural stem cells. In some embodiments, the differentiated cells are a mixed population of cells belonging to one or more desired lineages. In preferred embodiments of the invention, the differentiated cells are neural cells. As a specific example, they are neurons.

The differentiation cultures may involve more than one stage, with each stage using differing culture conditions. Such variation of culture conditions can be used to increase the overall yield of differentiated cells. Accordingly, in one embodiment of the invention, step d) comprises (i) incubating the culture under conditions conducive to the proliferation of the stem cells and (ii) incubating the culture under conditions conducive to the differentiation of the stem cells into the desired differentiated cells.

The methods for production of differentiated cells of the invention are generally carried out under similar conditions to those used for producing stem cells according to the first aspect of the invention.

Thus, the culture of step d) is preferably subjected to agitation, as described herein, and one or more of the oxygen concentration, the pH, the concentration of glucose, the concentration of lactate, and the shear rate are controlled.

Similarly, methods of the invention for producing differentiated cells preferably comprise inoculating the first volume of culture medium with ES cells or other stem cells in suspension. As discussed previously, the suspension may comprise single cells or clusters of stem cells, for example clusters of stem cells each containing from about 10 to about 20 or 30 cells.

In the methods of the invention for producing differentiated cells, step d) can comprise periodic replacement of a proportion of the medium volume with fresh medium, as described above. For example, the proportion of the medium volume can be 10%, 20%, 30%, 40%, 50%, 60%, 70%, 80% or 90% and the medium can be replaced every 12, 24, 36 or 48 hours. Continuous perfusion methods as described herein are also suitable for the production of differentiated cells.

Typically, the total culture volume is in excess of about 100 ml, 250 ml, 500 ml or 1000 ml.

The methods for producing differentiated cells according to the invention preferably comprise the step of e) harvesting the desired differentiated cells. Harvesting the differentiated cells may be carried out using the methods described for harvesting stem cells. Thus, harvesting may comprise a) isolating the microcarriers from the culture medium; and b) separating the differentiated cells from the microcarriers. As described above, the differentiated cells can be separated from the microcarriers using an enzymatic or a non-enzymatic cell dissociation reagent, for example trypsin-EDTA. In a preferred embodiment, the microcarriers are soluble, more preferably in the cell dissociation reagent. Optionally, the harvested cells, or a proportion thereof, are used to seed one or more further cultures as described herein. This step can be repeated, as desired, to create a continuous supply of differentiated cells.

The methods of the invention for producing differentiated cells can also be adapted for feeder-dependent cell types. Thus, in one embodiment feeder cells are permitted to adhere to the microcarriers prior to the inoculation of step a). Preferably, the feeder cells are permitted to proliferate until confluence prior to the inoculation of step a). It is also preferred for the feeder cells to be inactivated prior to the inoculation of step a).

The yield of stem cells or differentiated cells obtained using the methods of the invention will vary according to the culture conditions, culture volume and the initial and final cell types. However, in the specific examples production of populations of 5×10⁷-10⁸ and 1-1.5×10⁸ mouse ES cells (1-2×10⁶ and 2-3×10⁶ cells/ml). Similar yields of mouse neural stem cells have also been obtained using the methods of the invention. Preferably, the yield of stem cells or differentiated cells represents a 10-fold, 20-fold, 50-fold or even 100-fold or greater expansion of the originally inoculated cell population.

A further aspect of the invention provides a population of differentiated cells obtainable by the methods of the second and third aspects of the invention. Preferably, the population comprises at least about 10⁸ cells. Larger populations of cells can be obtained according to the invention, for example populations of at least 5×10⁸, 10⁹ or 10¹⁰ cells. In some embodiments, the population of cells is frozen using conventional cryopreservation techniques.

In a related aspect, the invention provides a composition comprising the differentiated progeny of stem cells attached to microcarriers. The differentiated progeny can be somatic stem cells, haematopoietic stem cells, epidermal stem cells, mesenchymal stem cells, adipose tissue-derived stem cells, muscle stem cells or neural stem cells, and may be a mixed population of cells belonging to one or more lineages. Preferably, the differentiated progeny are neural cells.

The invention also provides a composition comprising neural stem cells attached to microcarriers. The neural stem cells may, for example, be expanded using the methods of the first aspect of the invention or produced by differentiating ES cells according to the second or third aspect. Preferably, the composition comprises at least 10⁸, 5×10⁸, 10⁹ or 10¹⁰ cells.

A further aspect of the invention provides a kit comprising a population of stem cells or the differentiated progeny thereof attached to microcarriers and medium for the maintenance of the cells in culture. The cells may be supplied as actively growing cultures or may be frozen using conventional cryopreservation techniques. The cells may be supplied in any convenient format for further culture or other applications, including cells in vials, tissue culture plates, flasks or other suitable containers. In one embodiment, the cells are dispensed into multi-well plates ready for use in assays as described herein.

The ability of the methods of the invention to reproducibly generate large numbers of stem cells or differentiated cells of high quality opens up a number of possibilities for cell-based therapies. Accordingly, a further aspect of the invention provides a pharmaceutical composition comprising a population of stem cells or the differentiated progeny thereof attached to microcarriers. Preferably, the microcarriers used in this aspect of the invention are biocompatible and/or biodegradable. It is also desirable, in terms of obtaining regulatory approval, for the composition to be free of animal-derived components. Thus, for example gelatin microparticles and gelatin or collagen coatings are preferably avoided.

The cells produced according to the methods of the invention are particularly suitable for use in assays, e.g. in drug discovery applications. Thus, another aspect of the invention provides an assay reagent comprising a population of stem cells or the differentiated progeny thereof attached to microparticles.

A related aspect of the invention provides use of a population of stem cells or the differentiated progeny thereof attached to microcarriers in an assay for identifying factors that influence cell growth, survival and/or differentiation. The factors can be, for example, small molecules including new chemical entities, biological molecules including nucleic acids and proteins, or interactions between one or more molecules. Such interactions may, for example, involve molecular signalling processes that control proliferation, survival and/or differentiation, including ligand-receptor interactions.

In a related aspect, the invention provides an assay for identifying factors that influence cell growth, survival and/or differentiation comprising the steps of:
i) obtaining a population of stem cells or the differentiated progeny thereof attached to microcarriers;
ii) exposing a proportion of the stem cells or the differentiated progeny thereof to one or more factors; and
iii) determining the effect of the one or more factors on cell growth, survival and/or differentiation.

A number of assays suitable for determining the effects of factors on cell growth, survival and/or differentiation have been reported in the literature and are available to the operator of high-throughput screens.

The methods of the invention can also be adapted to provide structured populations of cells, e.g. bioartificial organs. Thus, a further aspect of the invention provides a method for producing a structured population of stem cells or the differentiated progeny thereof comprising:
a) inoculating a first volume of culture medium containing one or more three dimensional substratum supports with stem cells;
b) allowing the stem cells to adhere to the one or more substratum supports; and
c) incubating the culture under conditions conducive to the proliferation and/or differentiation of the stem cells

In some embodiments, the method comprises adding a second volume of medium to the culture after step b).

A number of suitable substratum supports have been described, for example in Xu and Reid (2001) Ann NY Acad Sci 944: 144-159. Preferably, the substratum supports are biocompatible and/or biodegradable. Such structured populations of cells have potential application, for example in the development of implants for production of insulin.

The invention will now be described in more detail in the following examples.

### EXAMPLE 1

### Microcarrier Culture of Mouse Embryonic Stem Cells

The passaging technique and cell harvest were designed for maintenance of cell number, viability and function, in order to produce large numbers of high quality cells. The protocols below detail microcarrier culture of mES cells and their harvest in either serum containing or serum free media (ESGRO Complete) and neural differentiation of mES in serum free media (RHB-A®) on microcarriers.

Protocol for Microcarrier Suspension Culture of mES Cells
1. Passage the inoculum cells 24 hrs prior to use such that the flasks are approximately 70% confluent for setting up the scale-up cultures.
2. Prepare the appropriate sized spinner flask (volume range 100 ml/250 ml/1000 ml). Typically follow a thorough wash and rinse procedure, followed by a rinse in deionised water. Let the flask dry and use a silicone coating e.g. Sigmacote (Sigma, UK) to minimise the tendency for cells/carriers to adhere to the sides of the flask. Sterilise the flask by autoclaving.
3. Weigh out the required amount of microcarriers and autoclave according to manufacturers instructions. If required microcarriers can be coated by immersion in sterile 0.1% gelatin solution prior to use.
4. After autoclaving, rinse the microcarriers with sterile PBS and allow to settle. Aspirate wash PBS and add a small volume of cell culture media (˜50 ml). Incubate at 37° C. for minimum 30 minutes. This step can be omitted if the microcarriers are coated in gelatin.
5. Dissociate and count the cells from the TC flask, ensuring a single cell suspension and suspend the required inoculum in sufficient media.
6. Remove the excess media from the microcarriers and add fresh culture media (warm), plus the cells and mix gently to disperse.
   a. Typical inoculation cell concentration is 5×10⁴ cells/ml final culture volume.
   b. Microcarrier density is dependent on carrier used. For

Cultispher gelatin (Percell Biolytica) microcarriers 0.5-1 g/litre final culture volume is typical.
7. Rinse the spinner flask with media to remove any residual liquid from autoclaving.
8. Add the microcarriers and the cell suspension to the stirrer flask. Add sufficient volume of culture media so that there is a 1 cm depth of media in the bottom of the spinner flask. Place on spinner base in an incubator at 37° C. and 7% CO₂, with the side caps loosened/adapted with air filters to allow for gas exchange. Allow the carriers and cells to settle, but gently agitate/spin the flask every two hours to evenly distribute the cells on the carriers.
9. After 24 hours add culture media to the final culture volume and spin continuously for the duration of the culture. Stirring speed should be sufficient to prevent settling of the carriers, but care should be taken that the cells are not exposed to excessive shear rates (typical spinning speed-40 rpm). Maximum shear stress values of 15-30 dyn/cm² have been reported to cause damage to cells attached to surfaces, and shear stresses approaching these values should therefore be avoided.
10. 50% of the media volume should be changed every 24 hours or as required.
11. Cell growth can be monitored by taking samples from the spinner flask and removing the cells using enzyme. The cells can then be counted. If the cells have a GFP reporter (e.g. Oct4GFP cell line), then the GFP signal can be used to give a visual indication of cell distribution on the carrier via fluorescent microscopy.

### Cell Harvest Protocol

1. Let microcarriers settle in flask for 5 minutes.
2. Aspirate excess media, taking care not to aspirate microcarriers.
3. Wash x×2 with warm PBS, letting the carriers settle between washes and aspirating the PBS.
4. After the final PBS wash, let the carriers settle and aspirate as much of the PBS as possible.
5. Add suitable enzyme (e.g. 0.1% trypsin solution) and spin gently. Cells should begin to detach within 2-3 minutes. If using a gelatin microcarrier, such as Cultispher, then continue until microcarriers dissolve completely.
6. Quench enzyme with serum containing media (or if serum free, dilute with serum free media) and collect cell suspension. If carriers are not dissolved then pass suspension through cell strainer (<100 micron) to separate cells from microcarriers.
7. Centrifuge cell suspension and resuspend in fresh media to remove remaining enzyme. Count cells and use to re-seed fresh microcarriers or differentiate as required.
8. Typical yields were in the range of 2-3×10⁶ cells/ml final culture volume.

### EXAMPLE 2

### Differentiation on Microcarriers

1. Follow protocols as for microcarrier culture and harvest of mES cells as set out in Example 1, but use RHB-A® (available from Stem Cell Sciences) rather than growth media.
2. There is no requirement to preseed the cells in 10% FCS media; cells can be seeded directly into RHB-A®.
3. Seeding density is the same as for growth conditions.
4. After 8 days, expected yield is 1-2×10⁶ cells/ml.
5. Cells can be replated and a high proportion of the live cell population is Sox-1 positive.

### EXAMPLE 3

### Microcarrier Culture and Differentiation of Mouse Neural Stem (mNS) Cells

Microcarrier Suspension Culture of mNS cells
1. Prepare the inoculum flask in RHB-AO media+EGF+FGF-2 (both at 20 ng/ml final concentration) so that cells are in log phase growth for seeding onto microcarriers.
2. Prepare the appropriate sized spinner flask (volume range 100 ml/250 ml/1000 ml). Typically follow a thorough wash and rinse procedure, followed by a rinse in deionised water. Let the flask dry and use a silicone coating e.g. Sigmacote (Sigma, UK) to minimise the tendency for cells/carriers to adhere to the sides of the flask. Sterilise the flask by autoclaving.
3. Weigh out the required amount of microcarriers and autoclave according to manufacturers instructions. If required microcarriers can be coated by immersion in sterile 0.1% gelatin solution prior to use.
4. After autoclaving, rinse the microcarriers with sterile PBS and allow to settle. Aspirate PBS and add a small volume of RHB-A® cell culture media+growth factors (˜20 ml). Incubate at 37° C. for minimum 30 minutes.
5. Dissociate and count the cells from the TC flask, ensuring a single cell suspension and suspend the required inoculum in sufficient media.
6. Remove the excess media from the microcarriers and add fresh culture media (warm), plus the cells and mix gently to disperse.
   a. Typical inoculation cell concentration is 5×10⁴-1×10⁵ cells/ml final culture volume.
   b. Microcarrier density is dependent on carrier used. For Cultispher gelatin (Percell Biolytica) microcarriers 0.5-1 g/litre final culture volume is typical. Other suitable microcarriers include Cytodexl/3 (Amersham Biosciences) used at 1 g/litre.
7. Rinse the spinner flask with media to remove any residual liquid from autoclaving.
8. Add the microcarriers and the cell suspension to the stirrer flask. Add sufficient volume of RHB-A® culture media+growth factors so that there is a 1 cm depth of media in the bottom of the spinner flask. Place on spinner base in an incubator at 37° C. and 7% CO₂, with the side caps loosened/adapted with air filters to allow for gas exchange. Allow the carriers and cells to settle, but gently agitate/spin the flask every two hours to evenly distribute the cells on the carriers.
9. After 24 hours add culture media to the final culture volume and spin continuously for the duration of the culture. Stirring speed should be sufficient to prevent settling of the beads, but care should be taken that the cells are not exposed to excessive shear rates (typical spinning speed-40 rpm).
10. 50% of the media volume should be changed every 24 hours or as required.
11. Cell growth can be monitored by taking samples from the spinner flask and removing the cells using enzyme. The cells can then be counted.

### Cell Harvest Protocol

1. Let microcarriers settle in flask for 5 minutes.
2. Aspirate excess media, taking care not to aspirate microcarriers.
3. Wash ×2 with warm PBS, letting the carriers settle between washes and aspirating the PBS.
4. After the final PBS wash, let the carriers settle and aspirate as much of the PBS as possible.
5. Add suitable enzyme (e.g. 0.1% trypsin solution) and spin gently in an incubator at 37° C. Cells should begin to detach within 2-3 minutes. If using a gelatin microcarrier, such as Cultispher, then continue until microcarriers dissolve completely.
6. Dilute enzyme with excess RHB-A® media and collect cell suspension. If carriers are not dissolved then pass suspension through cell strainer (<100 micron) to separate cells from microcarriers.
7. Centrifuge cell suspension and resuspend in fresh RHB-A® media+growth factors to remove remaining enzyme. Count cells and use to re-seed fresh microcarriers or replate and differentiate as required.
8. Typical yield is in the range of 1-2×10⁶ cells/ml final culture volume.

### Differentiation on Microcarriers

1. mNS cells can be differentiated on microcarriers by removal of growth factors from the cell culture media.
2. Stop spinning and allow microcarriers to settle.
3. Aspirate growth media (RHB-A®+EGF+FGF-2) and wash with 50 ml RHB-A® alone.
4. Aspirate wash RHB-A®.
5. Resuspend microcarriers in RHB-A® and continue to spin.
6. Cells will differentiate to neural cells over 3-7 days, yielding neural cells adhered to the microcarriers.
7. Neural cells are optionally removed from the microcarriers. The more differentiated the cells are the more fragile they become, and it may not be possible to remove the cells from the microcarriers using enzymatic methods.

### EXAMPLE 4

### Microcarrier Culture of Human Embryonic Stem (hES) Cells

As some hES cells are dependent on feeder cells, this protocol describes a method for co-culture of the hES cells on microcarriers with feeder cells.

Method 1: Expansion of feeder cells on microcarriers; feeder cells inactivated in situ:
1. Prepare the feeder cell inoculum in tissue culture flasks such that the cells are in log phase growth to seed onto the microcarriers.
2. Prepare the appropriate sized spinner flask (volume range 100 ml/250 ml/1000 ml). Typically follow a thorough wash and rinse procedure, followed by a rinse in deionised water. Let the flask dry and use a silicone coating e.g. Sigmacote (Sigma, UK) to minimise the tendency for cells/carriers to adhere to the sides of the flask. Sterilise the flask by autoclaving.
3. Weigh out the required amount of microcarriers and autoclave according to manufacturers instructions. If required microcarriers can be coated by immersion in sterile 0.1% gelatin solution prior to use.
4. After autoclaving, rinse the microcarriers with sterile PBS and allow to settle. Aspirate PBS and add a small volume of feeder cell culture media (˜50 ml). Incubate at 37° C. for minimum 30 minutes. This step can be omitted if the microcarriers are coated with gelatin.
   a. For HS27 foetal foreskin fibroblasts use 10% FCS in IMDM.
5. Dissociate and count the feeder cells from the TC flask, ensuring a single cell suspension, and suspend the required inoculum in sufficient media.
6. Remove the excess media from the microcarriers and add fresh culture media (warm), plus the cells and mix gently to disperse.
   a. Typical inoculation cell concentration is 1×10⁵ cells/ml final culture volume.
   b. Microcarrier density is dependent on the carrier used. For

Cytodexl/3 (Amersham Biosciences) use at 1 g/litre final culture volume.
7. Rinse the spinner flask with media to remove any residual liquid from autoclaving.
8. Add the microcarriers and the cell suspension to the stirrer flask. Add sufficient volume of feeder cell culture media so that there is a 1 cm depth of media in the bottom of the spinner flask. Place on spinner base in an incubator at 37° C. and 7% CO₂, with the side caps loosened/adapted with air filters to allow for gas exchange. Allow the carriers and cells to settle, but gently agitate/spin the flask every two hours to evenly distribute the cells on the carriers.
9. After 12-24 hours add culture media to the final culture volume and spin continuously. Stirring speed should be sufficient to prevent settling of the beads, but care should be taken that the cells are not exposed to excessive shear rates (typical spinning speed-40 rpm).
10. 50% of the media volume should be changed every 24 hours or as required.
11. Cell expansion can be monitored by taking a small sample from the spinner and staining with a nuclear stain such as Hoechst to visualise the cell coverage on the microcarrier.
12. Once feeder cells are deemed to have reached confluence, inactivate by γ-irradiation or mitomycin c treatment.

### Method 2: Expansion and Inactivation of Feeder Cells in TC Flask Prior to Seeding on Microcarriers

1. Prepare the feeder cells in tissue culture treated flasks until the required cell number is reached
   a. Seeding density should be evaluated for each feeder cell line in order to establish the number of cells required to form a confluent monolayer on the microcarrier as there will be no expansion post-inactivation.
2. Inactivate the feeder cells using γ-irradiation or mitomycin c treatment.
3. After careful washing, enzymatically remove the cells from the tissue culture flask using Accutase or trypsin/EDTA solution.
4. Resuspend the cells in serum containing feeder cell media.
5. Prepare the spinner flask and microcarrier as per steps 2-8 in Method 1.
6. Once cells have attached to the microcarriers, top up to final working volume and start spinning on spinner base in an incubator at 37° C. and 7% CO₂, with the side caps loosened/adapted with air filters to allow for gas exchange.

### Seeding of hES Cells on Microcarriers

1. Stop spinner flask and allow inactivated feeder cell coated microcarriers to settle.
2. Aspirate as much media as possible without removing microcarriers.
3. Wash ×2 with warm PBS.
4. Add 20% final volume of hES media and replace in incubator.
5. Remove media from flask of hES cells.
6. Wash ×2 with PBS.
7. Add enzyme or dissociation buffer such as Accumax to dissociate cells from tissue culture flask. Enzyme incubation time should be sufficient to dislodge cells from TC plastic and break colonies up into small clumps, but NOT to form a single cell suspension.
8. Transfer cells to Universal tube and dilute enzyme with fresh hES media.
9. Centrifuge at 0.2 rcf for 3 minutes.
10. Aspirate supernatant and resuspend cell pellet in fresh media.
11. Combine cell suspension with hES media and microcarriers in spinner flask in sufficient media to form a depth of 1 cm at the bottom of the spinner flask.
12. Place in incubator at 37° C. and 7% CO₂, with the side caps loosened/adapted with air filters to allow for gas exchange for 12-24 hours to allow the cells to attach to the microcarriers, gently agitating every 2 hours to ensure an even distribution of cells.
13. After 12-24 hours top up spinner flask to final working volume and begin spinning at the lowest rpm which will keep the microcarriers in suspension.
14. Change 50% media every 24 hours for the duration of the culture period.
15. Stirring speed may need to be increased over the duration of the culture period as the microcarriers have a tendency to form clumps.
16. Cells can be removed from microcarriers by enzymatic dissociation using collagenase or Accumax/Accutase. The hES cells can then be replated onto fresh feeders or differentiated as required.

The invention thus provides large scale production of stem cells, optionally on carriers.

1. A method for large-scale production of pluripotent stem cells, optionally embryonic stem (ES), embryonic carcinoma (EC), embryonic gonadal (EG), iPS or reprogrammed cells, comprising:
a) inoculating a first volume of serum-free culture medium containing a plurality of microcarriers with stem cells in suspension, wherein the suspension comprises clusters of stem cells;
b) allowing the stem cells to adhere to the microcarriers;
c) adding a second volume of serum-free medium to the culture; and
d) incubating the culture under conditions conducive to the proliferation of the stem cells.

2. A method for large-scale production of stem cells other than mouse ES cells comprising:
a) inoculating a first volume of culture medium containing a plurality of microcarriers with stem cells in suspension, wherein the suspension comprises clusters of stem cells;
b) allowing the stem cells to adhere to the microcarriers;
c) adding a second volume of medium to the culture; and
d) incubating the culture under conditions conducive to the proliferation of the stem cells.

3. The method of claim 2, wherein the culture medium is serum-free.

4. (canceled)

5. The method of claim 3, wherein one or more of the oxygen concentration, the pH, the concentration of glucose, the concentration of lactate, and the shear rate are controlled.

6-7. (canceled)

8. The method of claim 1, wherein the clusters of stem cells each contain from about 10 to about 30 cells.

9-11. (canceled)

12. The method of claim 1, wherein the total culture volume is in excess of about 500 ml.

13. (canceled)

14. The method of claim 1, comprising harvesting the cells by:
a) isolating the microcarriers from the culture medium; and
b) separating the stem cells from the microcarriers.

15. The method of claim 14, wherein the stem cells are separated from the microcarriers using an enzymatic or a non-enzymatic cell dissociation reagent and wherein the microcarriers are soluble in the cell dissociation reagent.

16-20. (canceled)

21. The method of claim 1, wherein feeder cells are permitted to adhere to the microcarriers prior to the inoculation of step a).

22. The method of claim 21, wherein the feeder cells are permitted to proliferate until confluence prior to the inoculation of step a).

23-24. (canceled)

25. A method for large-scale production of desired differentiated cells derived from stem cells comprising:
a) inoculating a first volume of culture medium containing a plurality of microcarriers with stem cells in suspension, wherein the suspension comprises clusters of stem cells;
b) allowing the stem cells to adhere to the microcarriers;
c) adding a second volume of medium to the culture; and
d) incubating the culture under conditions conducive to the differentiation of the stem cells into the desired differentiated cells.

26. The method of claim 25, wherein the culture medium is serum-free.

27. The method of claim 25, wherein the differentiated cells are somatic stem cells, haematopoietic stem cells, epidermal stem cells, mesenchymal stem cells, adipose tissue-derived stem cells, muscle stem cells or neural stem cells.

28. (canceled)

29. The method of claim 25, wherein the differentiated cells are neural cells.

30-34. (canceled)

35. The method of claim 25, wherein the clusters of stem cells each contain from about 10 to about 30 cells.

36-49. (canceled)

50. A composition comprising the differentiated progeny of stem cells attached to microcarriers.

51-52. (canceled)

53. The composition of claim 50, wherein the differentiated progeny are neural cells.

54. A composition comprising neural stem cells attached to microcarriers.

55-56. (canceled)

57. A pharmaceutical composition comprising a population of stem cells or the differentiated progeny thereof attached to microcarriers.

58-65. (canceled)
